(19) United States
(12) Patent Application Publication
(10) Pub. No.: US 2025/0001069 A1
(43) Pub. Date: Jan. 2, 2025

(54) INFUSION DEVICE WITH FIXATION OF THE FLUID LINE

(71) Applicant: FRESENIUS VIAL SAS, Brézins (FR)

(72) Inventors: Damien Archat, Grenoble (FR); Florie Perrigouard, La Tronche (FR)

(21) Appl. No.: 18/689,688

(22) PCT Filed: Sep. 7, 2022
(86) PCT No.: PCT/EP2022/074888

§ 371 Date: Mar. 6, 2024

(30) Foreign Application Priority Data
Sep. 10, 2021 (EP) 21315157.4

(51) Int. Cl. A61M 5/142

(52) U.S. Cl. CPC A61M 5/14216; A61M 5/14228

(57) ABSTRACT

An infusion device (1) for administering a medical fluid to a patient (P) comprises a housing (10) having a housing section, a receptacle (101) arranged on the housing (10) for receiving a flexibly deformable fluid line (2) longitudinally extending along a longitudinal direction of extension (D), and a pumping mechanism (4) configured to interact with the fluid line (2), the pumping mechanism (4) being configured to act onto the fluid line (2) for causing a fluid flow (F) through said fluid line (2). A fixation of a longitudinal portion (23) of the fluid line (2) with respect to the housing section is established in an operative state in which the fluid line (2) is received in the receptacle (101) for interacting with the pumping mechanism (4).

The invention relates to an infusion device for administering a medical fluid to a patient according to the preamble of claim 1, to a method for setting up an infusion operation using an infusion device and to an infusion line to be used with the infusion device.

An infusion device of this kind comprises a housing having a housing section, a receptacle arranged on the housing for receiving a flexibly deformable fluid line longitudinally extending along a longitudinal direction of extension, and a pumping mechanism configured to interact with the fluid line. The pumping mechanism is configured to act onto the fluid line for causing a fluid flow through the fluid line during operation of the infusion device. A housing section is a portion or a part of the housing.

An infusion device of this kind is generally denoted as a volumetric (peristaltic) infusion pump. The pumping mechanism herein in a peristaltic fashion acts onto a fluid line for transporting a medical fluid through the fluid line in order to deliver the medical fluid towards a patient.

A peristaltic infusion device as commonly known in the prior art for example comprises a pumping mechanism having a multiplicity of pump fingers for sequentially acting onto a fluid conduit in order to force fluid in a specified pumping direction through the conduit. Other peristaltic infusion devices use for example a wobble device which performs a wobbling action for acting onto a pump module comprising a membrane, the wobbling action causing a fluid to flow through the pump module.

There is a general desire to provide a peristaltic infusion device which may comprise a pumping mechanism of small size and easy construction, yet providing for a reliable and exact pumping action. Herein, it in particular is desirable to achieve a pumping action with a high accuracy of a pumped volume during an infusion operation, wherein a pumped volume should remain stable over successive pump cycles, with small deviations in the pumped volume in different cycles. The accuracy in the pumped volume per volume is important in particular for ensuring an infusion of a defined dosage of a medical fluid during an infusion operation such that a patient is administered that precise dosage that he or she is scheduled to receive.

WO 2012/049263 A1 discloses an infusion device comprising a pumping mechanism having a wobble mechanism for acting onto a pump module. A wobbling disc herein is connected at a slanted angle to a rotational shaft such that by rotating the shaft the wobbling disk can be moved to act onto a membrane of the pump module.

From WO 2010/088143 A1 a peristaltic infusion device is known which comprises a pumping mechanism having roller elements being placed on a common carrier element, the roller elements being configured to act onto a fluid conduit by rotating the carrier element in order to transport fluid through the fluid conduit.

It is an object of the instant invention to provide an infusion device and a method for setting up an infusion operation using an infusion device which may allow for a reliable and exact infusion operation.

This object is achieved by means of an infusion device comprising the features of claim 1.

Accordingly, the infusion device comprises a fixation of a longitudinal portion of the fluid line with respect to the housing section in an operative state in which the fluid line is received in the receptacle for interacting with the pumping mechanism to hold the longitudinal portion (23) of the fluid line (2) in place with respect to the housing section.

A pumping mechanism of the infusion device may for example comprise pump fingers, which are movable in a concerted fashion in order to act onto the fluid line received in the receptacle for pumping a fluid through the fluid line in a peristaltic pumping action. By moving the pump fingers the fluid line is locally depress, wherein a location of depression varies over a pump cycle such that fluid is transported through the fluid line.

The action of e.g. pump fingers of a pumping mechanism on the fluid line during operation of the infusion device causes strain and deformation on the fluid line. Friction conditions in the interaction between the pumping mechanism and the fluid line herein may vary over a pump cycle, such that the fluid line-in conventional setups-overall or locally may move, for example along the longitudinal direction of extension or along a transversal direction of extension, with respect to the housing section. It in this respect has been found that a pumped volume per cycle may vary from one cycle to another, due to varying conditions and due to a displacement of the fluid line within the receptacle. This affects the pumping accuracy, in that a pumped volume during one cycle may differ from a pumped volume in another cycle.

To overcome this effect and to improve pumping accuracy, it has been found that it may be beneficial to fix a portion of the fluid line with respect to the housing section such that a movement of the portion of the fluid line (other than the deformation caused by the pumping mechanism for transporting fluids through the fluid line) with respect to the housing section during operation of the infusion device is eliminated or at least reduced. For this, a fixation is provided which serves for fixing a longitudinal portion of the fluid line with respect to the housing section in an operative state in which the fluid line is received in the receptacle for interacting with the pumping mechanism.

The fixation in particular may serve to hold the longitudinal portion of the fluid line in place with respect to the housing section such that the longitudinal portion may not move in particular longitudinally along the longitudinal direction of extension of the fluid line with respect to the housing section, and/or in a direction transverse to the longitudinal direction of extension. The fluid line hence may be flexibly deformed by action of the pumping mechanism in order to transport fluid through the fluid line during operation of the infusion device, but overall is held in place along the longitudinal direction of extension or transverse to the longitudinal direction of extension during operation of the infusion device.

Because the fluid line is held in place, the pumping mechanism interacts with the fluid line in each cycle while the fluid line remains in place and is not moved from one cycle to another. This may enable to improve pumping accuracy, in that the pumping mechanism experiences the same conditions of the fluid line in each pump being cycle, such that the pumped volume remains stable from one cycle to another.

The fixation may for example be established by a gluing connection. In another embodiment, the fixation may be established by a positive-locking connection.

In one embodiment, the infusion device comprises a fixation member for establishing said fixation of the longitudinal portion of the fluid line with respect to the housing section. The fixation member may be a separate member with respect to the housing section. In the operative state the fixation member is fixedly connected to the housing section such that by means of the fixation member the longitudinal portion of the fluid line is held fixed with respect to the housing section.

In another embodiment, the fixation member may be integrally formed with the housing section such that the longitudinal portion of the fluid line is fixedly connected to the housing section.

By means of fixedly connecting the longitudinal portion of the fluid line to the fixation member, degrees of freedom for movement of the fluid line are reduced. In particular, by means of the fixation member the fluid line at its longitudinal portion is held in place with respect to the housing section of the housing such that the longitudinal portion may not longitudinally or transversely move on the housing section, a deformation of the fluid line at the longitudinal portion however being possible in order to cause a pumping action using the pumping mechanism.

In one embodiment, the fixation member is fixedly connected to a wall section of the fluid line along said longitudinal portion of the fluid line such that the wall section is held in place with respect to the housing section in said operative state. The fluid line is flexibly deformable such that the pumping mechanism may act onto the fluid line and may locally deform the fluid line in order to cause a peristaltic pumping action. The fluid line herein may have for example a circular cross section (in a non-deformed state) with a circular fluid conduit formed within the fluid line. The pumping mechanism herein may deform the fluid line such that the fluid conduit is locally occluded, wherein a location of occlusion may move longitudinally along the fluid line for causing a peristaltic pumping action and for pumping a defined volume during a pumping cycle.

By fixing a wall section of the fluid line to the fixation member, the fluid line with its wall section is fixedly connected and held in place with respect to the fixation member during a pumping action. Other sections of the fluid line however may flexibly be deformed with respect to the fixation member, such that a pumping action may be achieved.

In one embodiment, the longitudinal portion comprises a length measured along the longitudinal direction of extension, wherein the fixation member is fixedly connected to said wall section by means of a connection formed along said length. The longitudinal portion may in particular comprise a length matching or exceeding a length of a pumping mechanism, such that the fluid line by means of the fixation member is held in place in the region of the pumping mechanism. By forming a connection along the length of the longitudinal portion a line connection is established, by means of which the longitudinal portion of the fluid line is connected to the fixation member and thus is held in place with respect to the fixation member during a pumping action of the pumping mechanism.

The connection in particular may be formed by a gluing connection, a welding connection, a positive-fit connection or a force-fit connection. By means of such connection the longitudinal portion is tightly held on the fixation member, in particular by gluing or welding a wall section of the longitudinal portion to the fixation member, or by forming a positive-fit or force-fit connection between a wall section of the longitudinal portion and the fixation member.

In one embodiment, for forming a positive-fit connection in between the longitudinal portion of the fluid line and the fixation member the fluid line may comprise a connection element connected to (for example integrally formed on) the longitudinal portion and engaging with the fixation member. The connection element may for example engage with an undercut section formed on the fixation member such that a positive-fit connection in between the longitudinal portion of the fluid line and the fixation member is formed.

In another embodiment, the longitudinal portion of the fluid line may be integrally formed with the fixation member.

In one embodiment, the fixation member is formed by a rigid member extending longitudinally along the longitudinal portion of the fluid line. The rigid member may be integrally formed with the housing section of the housing, or may have the shape of a bar element to be received in a fixation receptacle of the housing section for fixing the bar element to the housing section. By means of the rigid member a stiffening of the fluid line in the region of the longitudinal portion is achieved, such that the longitudinal portion assumes a defined, in particular straight shape and may not be bent or lengthened out of that defined shape.

In another embodiment the fixation member could be flexibly deformable, at lea tina state prior to attaching the fixation member to the housing section. The fixation member may assume a sufficient stiffness for holding the longitudinal portion of the fluid line when it is attached to the hosing section. The fixation member, according to this embodiment may for example be formed by an adhesive tape, for example a two-sided adhesive tape, which on one side is adhesively connected to the fluid line and on another side is adhesively connected to the housing section.

In one embodiment, the fixation member comprises a connection face to which the longitudinal portion is connected. For example, a wall section of the fluid line may be fixedly connected to the connection face of the fixation member, for example by gluing or welding. The connection face may extend flatly along a plane spanned by the longitudinal direction of extension of the fluid line and a transverse direction, wherein the fluid line, when being flexibly deformed by the pumping mechanism for causing a pumping action, may abut with the connection face such that the fluid line may be locally depressed by means of the pumping mechanism with respect to the connection face of the fixation member.

In one embodiment, herein, the fixation member may comprise one or two bounding elements which protrude with respect to the connection face. The bounding elements may have the shape of ridges extending longitudinally along the connection face and bounding the connection face when viewed in a direction transverse to the longitudinal direction of extension, such that the fixation member at its front face assumes the shape of a cradle-like structure. The bounding elements in particular may provide bounding walls sideways of the front face, such that a transverse movement of the fluid line transverse to its longitudinal direction of extension is prohibited and bound by the bounding elements.

In one embodiment, the housing of the infusion device comprises a closure element, also denoted as door, arranged such that it is pivotable with respect to a front face of the housing. For placing a fluid line on the receptacle formed on the housing, the closure element may be opened, such that the receptacle is accessible, and a fluid line may be placed within the receptacle. For bringing the infusion device into an operative state, the closure element is closed such that the fluid line is received in between the closure element and the front face formed on the housing. Herein, the closure element may form the housing section relative to which the fluid line is fixed by means of the fixation member during operation of the infusion device. The longitudinal portion of the fluid line hence, during operation of the infusion device, is held fixed with respect to the closure element, such that a movement of the infusion line with respect to the closure element in the operative state of the infusion device is at least limited.

In another embodiment, the housing section is formed by a face of a main housing body of the infusion device, for example by a front face on which the receptacle for receiving the fluid line is formed.

In another aspect, a method for setting up an infusion operation using an infusion device for administering a medical fluid to a patient comprises: arranging a flexibly deformable fluid line in a receptacle arranged on a housing of the infusion device, the fluid line longitudinally extending along a longitudinal direction of extension; and, by arranging the fluid line in such a receptacle, bringing the fluid line in operative connection with a pumping mechanism of the infusion device configured to interact with the fluid line, the pumping mechanism being configured to act onto the fluid line for causing a fluid flow through said fluid line. Herein, the method includes the further step of fixing a longitudinal portion of the fluid line with respect to a housing section of the housing such that the longitudinal portion is held in place with respect to the housing section in an operative state in which the fluid line is received in the receptacle for interacting with the pumping mechanism.

In the scope of the invention is also proposed an infusion line for conveying a medical fluid for use with the above defined infusion device, the infusion line comprising the fluid line and, at least in part, the fixation member. The infusion line may be a kit with a standard fluid line associated with a fixation member to be inserted onto the infusion device or may be an integrated fixation member into or attached to the fluid line. It may also be possible that a first fixation member is part of the infusion line and a second fixation member is a part of the infusion device.

The advantages and advantageous embodiments as described above for the infusion device equally apply also to the method, such that it shall be referred to the above in this respect.

The idea underlying the invention shall subsequently be described in more detail with reference to the embodiments shown in the figures. Herein:

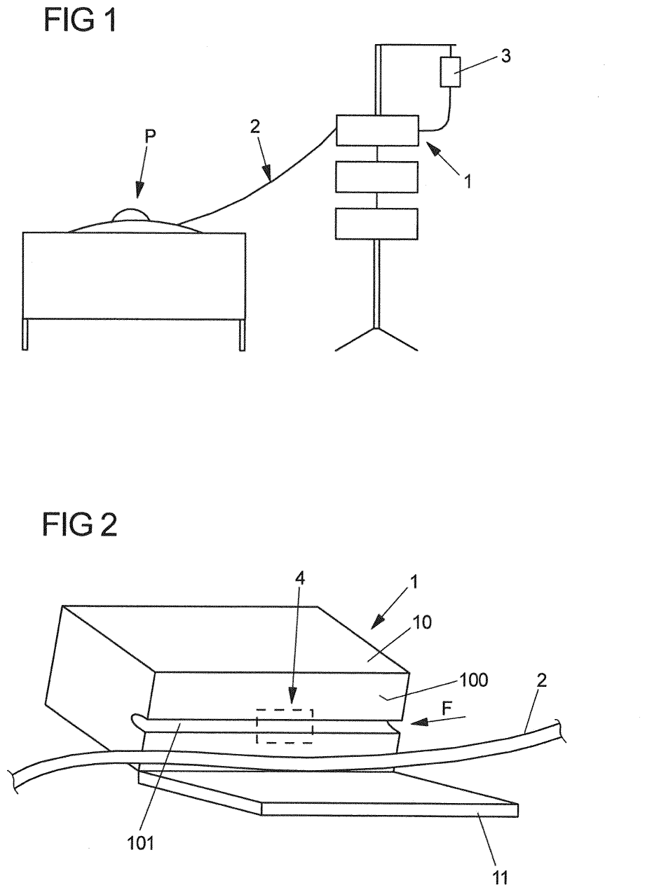

FIG. 1 shows a schematic drawing of an arrangement of infusion devices at the bedside of a patient;

FIG. 2 shows a schematic drawing of an infusion device configured as a volumetric (peristaltic) infusion device;

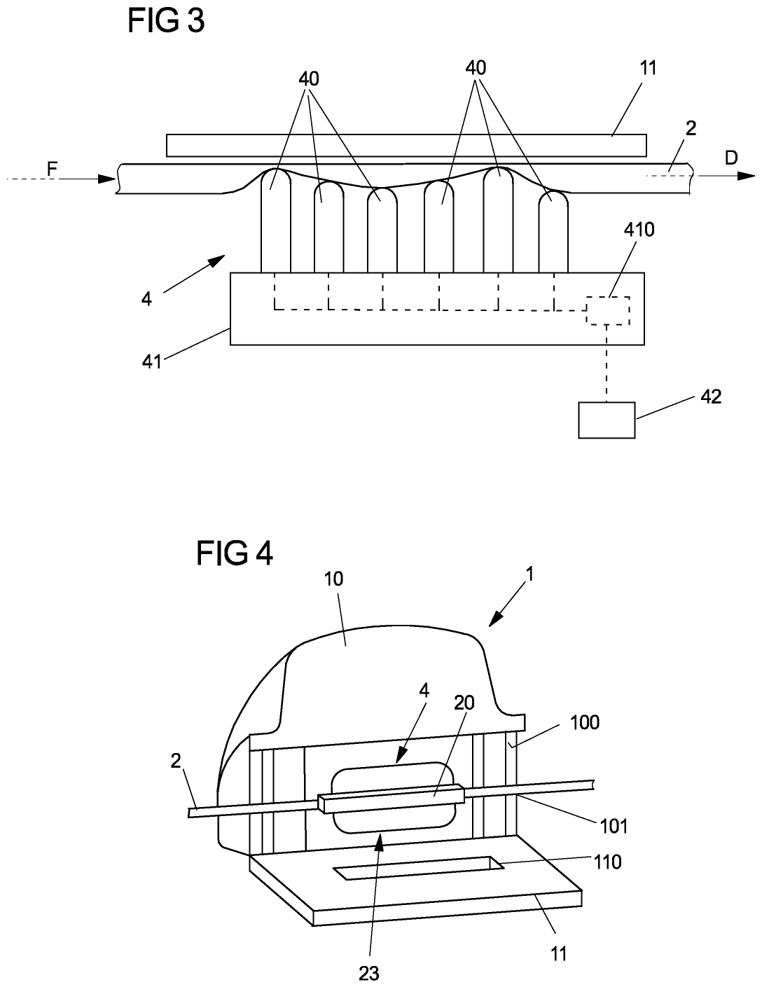

FIG. 3 shows a schematic view of a pumping mechanism of an infusion device for causing a peristaltic pumping action on a fluid line;

FIG. 4 shows a schematic view of an infusion device with a fluid line connected to a fixation member;

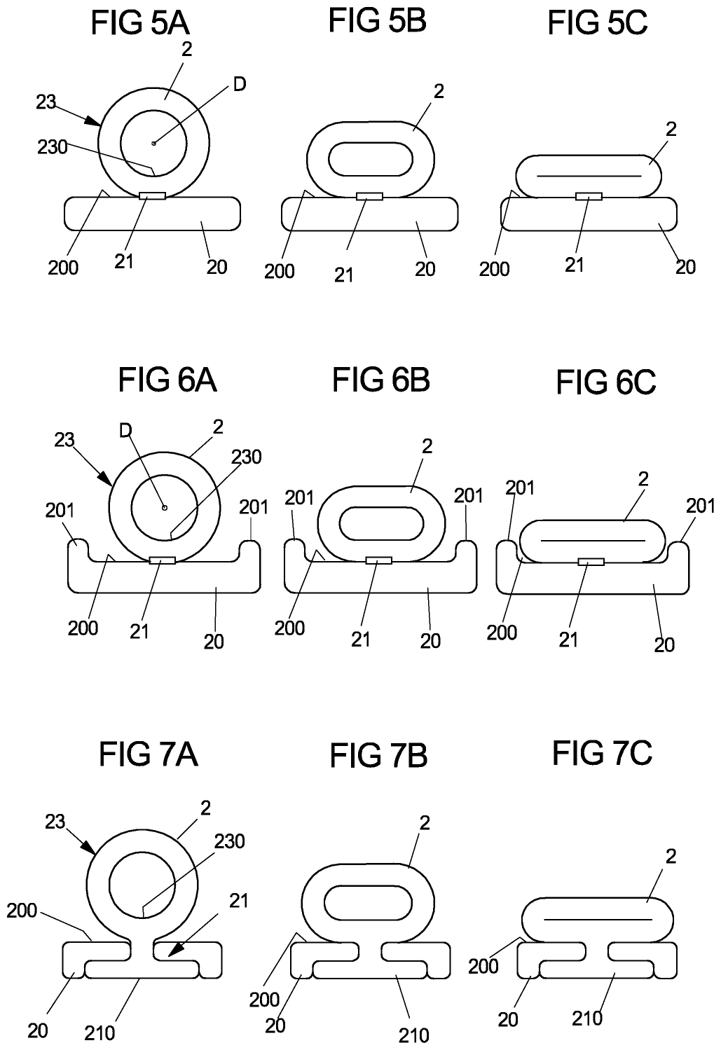

FIG. 5A shows an embodiment of an infusion line connected to a fixation member in a non-deformed state;

FIG. 5B shows the arrangement of FIG. 5A, during deformation of the fluid line;

FIG. 5C shows the arrangement of FIG. 5A, with the fluid line in a depressed, occluded state;

FIG. 6A shows another embodiment of an infusion line connected to a fixation member in a non-deformed state;

FIG. 6B shows the arrangement of FIG. 6A, during deformation of the fluid line;

FIG. 6C shows the arrangement of FIG. 6A, with the fluid line in a depressed, occluded state;

FIG. 7A shows yet another embodiment of an infusion line connected to a fixation member in a non-deformed state;

FIG. 7B shows the arrangement of FIG. 7A, during deformation of the fluid line;

FIG. 7C shows the arrangement of FIG. 7A, with the fluid line in a depressed, occluded state;

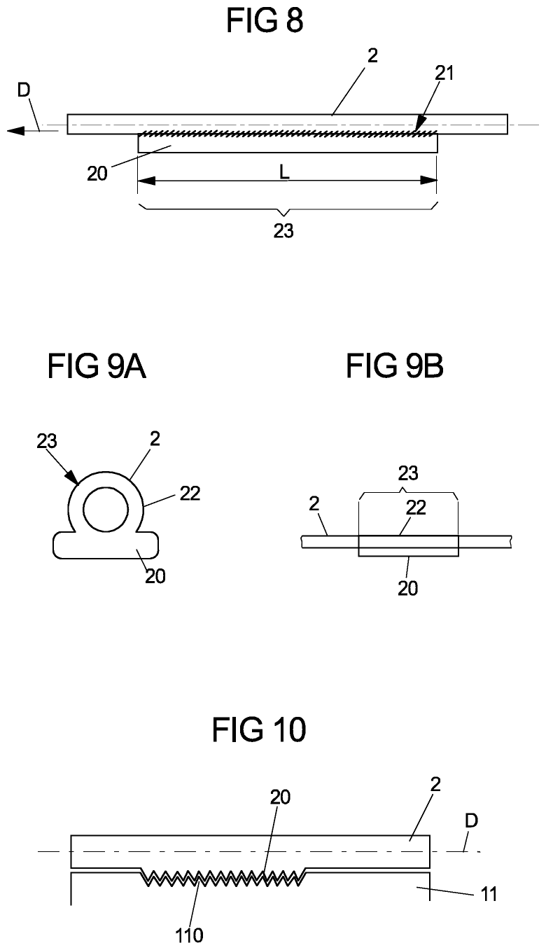

FIG. 8 shows a schematic view of a longitudinal portion of an infusion line connected to a fixation member;

FIG. 9A shows yet another embodiment of a fluid line connected to a fixation member;

FIG. 9B shows a schematic, longitudinal view of the arrangement of FIG. 9A;

FIG. 10 shows another embodiment of a fluid line connected to a fixation member; and

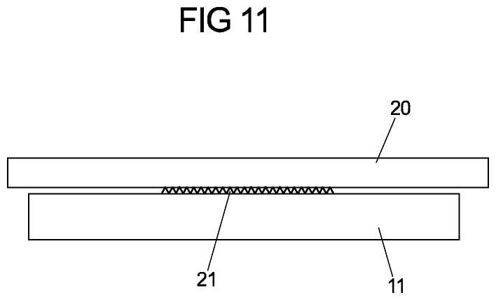

FIG. 11 shows another embodiment of a fluid line connected by a fixation to a housing section.

In a general setup, as illustrated in FIG. 1, a multiplicity of infusion devices 1 may be arranged at the bedside of a patient P for delivering one or multiple medical fluids, such as medications, nutritional liquids, a saline solution or the like, to the patient P. The infusion devices 1 may be arranged on a stand and each serve to deliver a medical fluid from a container 3 via a fluid line 2 towards the patient P.

An infusion device 1 as concerned herein in particular is configured as a volumetric (peristaltic) infusion pump for performing a peristaltic pump action for transporting a fluid through a fluid line 2.

As schematically shown in FIG. 2, an infusion device 1 of this type generally may comprise a housing 10 having a front face 100 on which a receptacle 101 for receiving a fluid line 2 is formed. A closure element 11 in the shape of a pivotable door may be connected to a main housing portion of the housing 10 and may be closed in order to securely receive and fasten a fluid line 2 within the receptacle 101 formed on the front face 100 of the housing 10.

Both the closure element 11 and the front face 100, in the context of this text, form housing sections of the housing 10.

The infusion device 1 comprises a pumping mechanism 4 for acting onto the fluid line 2. The pumping mechanism 4 generally is configured to act onto the fluid line 2 in a peristaltic moving manner in order to force a fluid in a flow direction F through the fluid line 2 such that fluid is pumped through the fluid line 2 generally from a container 3 towards a patient P.

Referring now to FIG. 3, in one embodiment the pumping mechanism 4 comprises pump elements 40 in the shape e.g. of pump fingers which are movable by a driving mechanism 41 comprising an electric drive motor 410 for moving the pumping elements 40 in a concerted fashion for causing a peristaltic pump action on a fluid line 2 received in between the pumping mechanism 4 and the closure element 11. In particular, using the pump elements 40 the fluid line 2 may be locally depressed for locally occluding the fluid line 2, wherein a location of occlusion is longitudinally moved along the fluid line by moving the pump elements 40 in order to cause a peristaltic pump action on the fluid line 2.

The infusion operation herein is controlled using a control device 42, which in particular is in operative connection with the drive motor 410 in order to control the infusion operation.

Referring now to FIG. 4, the closure element 11 in the shape of the door is pivotably connected to a main housing portion of the housing 10 of the infusion device 1 such that the closure element 11 is pivotably movable with respect to the front face 100 of the housing 10 on which the receptacle 101 is formed and on which a fluid line 2 is to be received for causing a pumping action on the fluid line 2.

For setting up an infusion operation, the fluid line 2 is placed within the receptacle 101, and the closure element 11 is closed. A longitudinal portion 23 of the fluid line 2 herein comes to rest in a region of the pumping mechanism 4, wherein the longitudinal portion 23 is brought into operative connection with the pumping mechanism 4, in particular the pump elements 40 of the pumping mechanism 4, when closing the closure element 11.

During a pumping action, the pump elements 40 act onto the fluid line 2 and locally depress and release the fluid line 2 in an alternating, concerted fashion in order to cause a peristaltic pump action. This causes a local deformation and strain on the fluid line 2, and also causes a varying friction on the fluid line 2 by interaction with the pump elements 40.

It has been found that the action of the pump elements 40 on the flexibly deformable fluid line 2 (which for example is made from a flexibly deformable PVC material or the like) may cause a movement of the fluid line 2 with respect to the front face 100 and the closure element 11, in particular along a longitudinal direction of extension D of the fluid line 2 and in a transverse direction transverse to the longitudinal direction of extension D. Such movement may cause differing conditions in the pumping action of the pumping mechanism 4, due to a displacement of the fluid line 2 with respect to the pumping mechanism 4, such that a pumped volume may vary from one pumping cycle to another. This may reduce the accuracy in the pumping action, in particular with respect to the reproducibility of a pumped volume from one cycle to another.

In order to improve the accuracy during an infusion operation, it is proposed to use a fixation member 20 for fixing the fluid line 2 with respect to the closure element 11 such that the fluid line 2, in particular in the region of its longitudinal portion 23, is held in place with respect to the closure element 11 forming a housing section and in this way the housing 10 during operation of the infusion device 1.

As visible from FIG. 4, in one embodiment the fixation member 20 may have the shape of a rigid bar element, which is to interact with a counter element in the shape of a fixation receptacle 110 formed on the inside of the closure element 11 for receiving the fixation member 20. When the closure element 11 is closed, the fixation member 20 comes to rest in the fixation receptacle 110 and hence is fastened to the closure element 11, wherein the fixation member 20 is fixedly connected to the longitudinal portion 23 of the fluid line 2 and hence provides for a fixation of the fluid line 2 in the region of the longitudinal portion 23 with respect to the closure element 11.

The fluid line 2 may be connected to the fixation member 20 in different ways. For example, a gluing connection, a welding connection, a positive-fit connection or a force-fit connection may be formed in between the fluid line 2 and the fixation member 20.

In one embodiment shown in FIGS. 5A to 5C, a wall section 230 of the longitudinal portion 23 of the fluid line 2 is fixedly connected to a fixation member 20 in the shape of a rigid bar element by means of a connection 21, for example formed by a gluing or welding connection. The fixation member 20 herein forms a flat connection face 200 to which the wall section 230 of the longitudinal portion 23 is fixedly connected such that the fluid line 2 at its wall section 230 is held in place on the fixation member 20. As visible in the sequence of FIGS. 5A to 5C, other portions of the fluid line 2 herein remain flexibly deformable, such that the fluid line 2 within its longitudinal portion 23 may be locally deformed in order to cause a pumping action on the fluid line 2.

Whereas in the embodiment of FIGS. 5A to 5C the fixation member 20 is flat at its connection face 200, in the embodiment of FIGS. 6A to 6C the fixation member 20 assumes a cradle-like structure in which the connection face 200-to which the wall section 230 of the longitudinal portion 23 of the fluid line 2 is connected-is bound by bounding elements 201 along a direction transverse to the longitudinal direction of extension D of the fluid line 2. The bounding elements 201 may have the shape of longitudinally extending ridges extending sideways on the front face 200 and protruding from the front face 200, such that a transverse movement of the fluid line 2 in a transverse direction with respect to the longitudinal direction of extension D is limited.

Whereas in the embodiments of FIGS. 5A to 5C and 6A to 6C the fluid line 2 is connected to the fixation member 20 by means of a gluing or welding connection 21, in the embodiment of FIGS. 7A to 7C the fluid line 2 is connected to the fixation member 20 by means of a positive-fit connection 21. For this, a connection element 210 in the shape of a positive-locking web extending along the longitudinal portion 23 of the fluid line 2 is in engagement with an undercut section of the fixation member 22 and thus establishes a positive-fit connection. In addition, a gluing or welding connection may be established, for example in between the connection element 210 and the fixation member 20.

Referring now to FIG. 8, in any of the embodiments of FIGS. 5A to 5C, 6A to 6C or 7A to 7C the connection 21 may be formed along the entire length L of the longitudinal portion 23, as it is schematically shown in FIG. 8. In another embodiment it however also is conceivable that the connection 21 is formed only along a portion of the fixation member 20, or at multiple points or sections which are displaced with respect to one another along the fixation member 20.

Referring now to FIGS. 9A, 9B, in another embodiment the longitudinal portion 23 of the fluid line 2 may be integrally formed with the fixation member 20, wherein the fixation member 20 may have an increased rigidity with respect to a flexible section 22 forming the longitudinal portion 23 of the fluid line 2.

As described with respect to FIG. 4, the fixation member 20 may interact with a counter element formed on the closure element 11 in order to provide for a fixation of the fluid line 2 on the closure element 11. For this, as described above, the fixation member 20 may be received in a receptacle 110 of the closure element 11.

In another embodiment, as shown in FIG. 10, the fixation member 20 may for example have the shape of a toothing or another positive-locking structure which may be brought into engagement with a corresponding counter structure on the closure element 11 in order to achieve a fixation of the fluid line 2 on the closure element 11, in particular along the longitudinal direction of extension D of the fluid line 2 with respect to the closure element 11.

In another embodiment, as shown in FIG. 11, the fluid line 2 is glued by a glue connection 21 directly to the closure element 11. For instance, the fluid line 2 may be prepared, prior to its installation in the infusion device 1, with a dedicated strip of glue or a sticker.

In yet another embodiment, a dedicated fluid line 2 may be obtained with internal wires having high elastic modulus (for instance Kevlar) co-extruded in the tube in order to limit the deformation along the pumping direction.

It has been shown in measurements that an improvement in accuracy of a pumped volume can be achieved by using a fixation of the flexibly deformable fluid line 2 with respect to the closure element 11.

The idea underlying the invention is not limited to the embodiments described above, but may be implemented also in an entirely different fashion.

An infusion device as described herein may be used to deliver different medical fluids, such as medication, nutritional liquid or the like, towards a patient.

The pumping mechanism of the infusion device acts onto a fluid line and may comprise pump elements in the shape of pump fingers, wherein also other pump mechanisms are conceivable.

A housing section to which the fluid line, in an operative state, is fixed by a fixation may be the closure member or the front face.

### LIST OF REFERENCE NUMERALS

1 Infusion device
10 Housing
100 Front face
101 Receptacle
11 Closure element
110 Counter member (fixation receptacle)
2 Fluid line
20 Fixation member
200 Face
201 Bounding element (ridge)
21 Connection
210 Connection element
22 Flexible section
23 Portion
230 Wall section
3 Container
4 Pumping mechanism
40 Pump elements (pump fingers)
41 Driving mechanism
410 Drive motor
42 Control device
Q D Longitudinal direction of extension
F Flow direction
L Length
P Patient

1. An infusion device for administering a medical fluid to a patient, comprising:
a housing having a housing section,
a receptacle arranged on the housing for receiving a flexibly deformable fluid line longitudinally extending along a longitudinal direction of extension,
a pumping mechanism configured to interact with the fluid line, the pumping mechanism being configured to act onto the fluid line for causing a fluid flow through said fluid line, and
a fixation of a longitudinal portion of the fluid line with respect to the housing section in an operative state in which the fluid line is received in the receptacle for interacting with the pumping mechanism to hold the longitudinal portion of the fluid line in place with respect to the housing section.

2. The infusion device according to claim 1, wherein the fixation is configured to hold the longitudinal portion of the fluid line in place with respect to the housing section along the longitudinal direction of extension and/or along a direction transverse to the longitudinal direction of extension.

3. The infusion device according to claim 1, wherein said fixation is established by a gluing connection.

4. The infusion device according to claim 1, further comprising a fixation member for establishing said fixation of the longitudinal portion of the fluid line with respect to the housing section.

5. The infusion device according to claim 4, wherein the fixation member is fixedly connected to a wall section of the fluid line along said longitudinal portion of the fluid line such that the wall section is held in place with respect to the housing section in said operative state.

6. The infusion device according to claim 5, wherein said longitudinal portion of the fluid line comprises a length measured along the longitudinal direction of extension, wherein the fixation member is fixedly connected to said wall section by means of a connection formed along said length.

7. The infusion device according to claim 6, wherein the connection formed along said length is formed by a gluing connection, a welding connection, a positive-fit connection, or a force-fit connection.

8. The infusion device according to claim 4, wherein the fluid line comprises a connection element connected to said longitudinal portion and engaging with the fixation member for forming a positive-fit connection between the longitudinal portion and the fixation member.

9. The infusion device according to claim 4, wherein the fixation member is formed by a rigid member extending longitudinally along said longitudinal portion of the fluid line.

10. The infusion device according to claim 9, wherein said rigid member is formed by a bar element to be received in a fixation receptacle of said housing section for fixing the bar element to said housing section.

11. The infusion device according to claim 4, wherein the fixation member comprises a connection face to which said longitudinal portion is connected.

12. The infusion device according to claim 11, wherein the fixation member comprises at least one bounding element protruding with respect to said connection face and bounding said connection face when viewed in a direction transverse to the longitudinal direction of extension.

13. The infusion device according to claim 1, wherein the housing comprises a closure element which is pivotable with respect to a front face of the housing, the closure element forming said housing section.

14. The infusion device according to claim 13, wherein the receptacle is formed on the front face for receiving the fluid line between the front face and the closure element.

15. A method for setting up an infusion operation using an infusion device for administering a medical fluid to a patient, comprising:
arranging a flexibly deformable fluid line in a receptacle arranged on a housing of the infusion device, the fluid line longitudinally extending along a longitudinal direction of extension;
by arranging the fluid line in said receptacle, bringing the fluid line in operative connection with a pumping mechanism of the infusion device configured to interact with the fluid line, the pumping mechanism being configured to act onto the fluid line for causing a fluid flow through said fluid line; and
fixing a longitudinal portion of the fluid line with respect to a housing section of the housing such that the longitudinal portion is held in place with respect to the housing section in an operative state in which the fluid line is received in the receptacle for interacting with the pumping mechanism.

16. An infusion line for conveying a medical fluid for use in an infusion device according to claim 4 wherein the infusion line comprises the fluid line and, at least in part, the fixation member.
